(19) United States
(12) Patent Application Publication
(10) Pub. No.: US 2012/0321612 A1
(43) Pub. Date: Dec. 20, 2012

(54) SYSTEMS AND METHODS FOR TREATING POST-OPERATIVE, ACUTE, AND CHRONIC PAIN USING AN INTRA-MUSCULAR CATHETER ADMINISTRATED COMBINATION OF A LOCAL ANESTHETIC AND A NEUROTOXIN PROTEIN

(76) Inventors: Gerardo Zavala, JR., San Antonio, TX (US); Gerardo Zavala, SR., San Antonio, TX (US)

(21) Appl. No.: 13/588,232

(22) Filed: Aug. 17, 2012

Related U.S. Application Data
(62) Division of application No. 12/689,381, filed on Jan. 19, 2010.
(60) Provisional application No. 61/145,621, filed on Jan. 19, 2009.
(60) Provisional application No. 61/257,724, filed on Nov. 3, 2009.
(60) Provisional application No. 61/295,960, filed on Jan. 18, 2010.

(51) Int. Cl. A61K 38/02; A61P 25/04; A61P 23/02; A61K 38/48

(52) U.S. Cl. 424/94.67; 514/18.3

(57) ABSTRACT

Systems, and methods for the use of such systems, are described that allow for the administration of a combination of a sustained release local anesthetic compound (such as bupivicaine) through a catheter based administration device and direct visualization or percutaneous injection of a neurotoxic protein compound (such as botulinum toxin) for post-operative and refractory treated muscle pain and discomfort in patients having undergone spinal surgery and other muscle splitting or treatments aimed at improving muscle pain. The systems utilize specific catheter-based administration protocols and methods for placement of the catheter in association with muscles surrounding the spine and other anatomical sites within the patient. The utilization of an initial bolus of a specific combination of medications (local anesthetic compound and\or neurotoxic protein compound) followed by a dosage pump administration through the catheter is anticipated. A variety of local anesthetics (in addition to bupivicaine) and a number of different neurotoxin proteins (in addition to the Botulinum toxin) may be utilized in the medicament administration protocols described.

This application claims priority to U.S. Provisional Patent Application Ser. No. 61/145,621, filed Jan. 19, 2009, entitled SYSTEMS AND METHODS FOR TREATING POST-OPERATIVE, ACUTE, AND CHRONIC PAIN USING AN INTRA-MUSCULAR CATHETER ADMINISTERED COMBINATION OF ANESTHETIC AND A NEUROTOXIN PROTEIN, and U.S. Provisional Patent Application Ser. No. 61/257,724, filed Nov. 3, 2009, entitled SYSTEMS AND METHODS FOR TREATING POST-OPERATIVE, ACUTE, AND CHRONIC PAIN USING AN INTRA-MUSCULAR CATHETER ADMINISTERED COMBINATION OF ANESTHETIC AND A NEUROTOXIN PROTEIN, and U.S. Provisional Patent Application Ser. No. 61/295,960, filed Jan. 18, 2010, entitled DECREASING POST OPERATIVE PAIN, the entire content of each is hereby incorporated by reference.

### FIELD OF THE INVENTION

The present invention relates generally to methods for treating muscle pain related to spasm, post operative and other types of muscle pain. The present invention relates more specifically to systems and methods for treating post-operative and chronic pain using a subfacial and intra-muscular catheter administered local anesthetic and injection of a neurotoxin protein.

### BACKGROUND

There is a need for the more effective administration of pain relief medication to two types of patients: 1) those who have undergone surgical and other muscle splitting procedures, especially those involving the spine and surrounding tissue (i.e. post-operative patients); and 2) those patients who have been refractory to current conventional treatments to alleviate muscle pain (i.e. chronic pain sufferers). At present, most treatment protocols call for the intermittent, or periodic, administration of local anesthetic compounds to the surgical site or area of pain in order to alleviate pain as it arises in a postoperative or conventionally-treated chronic muscular pain patient. In some cases, systems have been developed for the administration of a sustained release of a local anesthetic compound, such as through the use of a medication infusion catheter. A typical example of such an existing infusion catheter directed toward supra and subfacial administration of a local anesthetic or a narcotic is provided by the ON-Q® Painball infusion catheter system developed and marketed by I-Flow Corporation of Lake Forest, Calif. (hereinafter, the On Q System). Other efforts attempt to treat post-operative pain with periodic administrations (injections) of local anesthetic compounds and/or the use of local and systemic analgesics.

In post-operative patients, the choices of pain relief medications are aimed only at treating the multiple sources of the pain felt by most post-operative patients. This is not always effective. The trauma caused by a surgical procedure can result in pain derived directly from the site of intervention and from the indirect involvement of the surrounding tissue, especially the surrounding muscle. Surgical pain and trauma is experienced not only by those muscles directly cut at the surgical site but also indirectly to the surrounding muscles that tend to contract and/or stress in an effort to compensate for those muscles directly weakened by the surgical procedure.

Therefore, while local anesthetics and general analgesics can address pain associated with the post-operative patient in the acute pain setting (4-6 hours post operative), they often fail to fully address the ongoing pain that follows the 4-6 hours directly post-surgery, especially surgery involving the muscles attached to the spine and other muscle splitting procedures. Therefore, it becomes necessary to repeat administration of these pain relievers (local anesthetics and/or systemic analgesics and/or muscle relaxants) for extended periods of time, and for longer than what might be preferred. This is necessary because these medications alone fail to address one or more of the ongoing causes of post-operative pain, including that muscle spasm and pain deriving from the direct and indirect trauma discussed above.

For chronic muscular pain patients, current treatments available are comprised of one or a combination of the following: over-the-counter (“OtC”) medications, prescription medication, occupational/physical therapy, massage treatments, and invasive anesthetic delivery treatments to the epidural space, facet, or intramuscular spaces and other regionally affected areas. Although most patients' symptoms are improved with these interventions there remains a number of patients that would benefit from the introduction of a combination of catheter administration of anesthetic with neurotoxin protein.

Local anesthetics are agents which prevent transmission of nerve impulses without causing unconsciousness. Local anesthetics typically act by binding to fast sodium channels from within (in an open state). Local anesthetics can be either ester or amide based, or combination amine/ester based or natural anesthetics like saxitoxin and tetrodotoxin. Ester local anesthetics (for example, procaine, amethocaine, cocaine are generally unstable in solution but are fast-acting. Unfortunately allergic reactions are common with ester local anesthetics. Amide local anesthetics (for example, lidocaine, prilocaine, bupivicaine, levobupivacaine, ropivacaine and dibucaine) are generally heat-stable, with a long shelf life (around 2 years). They have a slower onset and longer half-life than ester anesthetics. These agents are generally used within regional and epidural or spinal techniques, due to their longer duration of action, which can often provide adequate analgesia for surgery, labor, and symptomatic relief.

Lidocaine (as an example) is a common local anesthetic and antiarrhythmic drug and is often used topically to relieve itching, burning and pain from skin inflammations, as well as being injected as a dental anesthetic, and in minor surgery. Lidocaine, the first amino amide type local anesthetic, was first synthesized in 1943 and was first marketed in 1948. As an anesthesia, lidocaine alters depolarization in neurons by blocking the fast voltage gated sodium (Na⁺) channels in the cell membrane. With sufficient blockade, the membrane of the presynaptic neuron will not depolarize and so fail to transmit an action potential, leading to its anesthetic effects.

While conventional pain management systems as described above may benefit from the sustained release of a local anesthetic, such systems fail to fully and efficiently address the pain associated with muscle splitting surgical intervention procedures in a post-operative patient or in those patients who have refractory pain to current treatments to help alleviate pain. A more complete combination of pain treatment compounds, administered in an effective manner, would serve to more efficiently and effectively address the pain and discomfort of a patient having undergone surgical or other related invasive procedures, and it would serve as an alternative to current treatments of chronic pain.

Most muscular pain, in particular refractory treated muscle pain and post-operative pain, is often comprised of two or more components, including incisional pain and muscle spasm. Severe pain can last for greater than 7 days if proper measures are not taken to address it. Long periods of post-operative pain and refractory muscular pain increase hospitalization, worsen patient outcomes and elevate total medical costs.

Common conventional pain treatments include opioids, non-steroidal anti-inflammatory drugs (NSAIDs), and muscle relaxants, and multimodal analgesic treatments are considered superior to monotherapy in improving pain. Preferred routes of delivery for analgesics include intravenous, patient-controlled analgesia (PCA) bolus, oral, intramuscular, and epidural.

Conventional post-operative and chronic pain treatments have several disadvantages, impacting the gastrointestinal (GI) system (constipation, post op illus, nausea, GI bleed), the respiratory system (atelectasis, pneumonia, pulmonary emboli), neurological system (altered mental status), and cardiovascular system (hypotension, tachycardia, myocardial infarction). In addition, prolonged periods of immobility can cause deep venous thrombosis, pneumonia, pain, and spasm. For these reasons, conventional post-operative treatments can prolong hospital stays and increase healthcare costs.

One third of all patients who underwent spinal surgery experienced severe bouts of nausea and vomiting post-operatively, prolonging hospital stays. Post-operative nausea and vomiting (PONV) can cause electrolyte imbalance, dehydration, general malaise, and can require prolonged physical therapy intervention. PONV is worsened by conventional post-operative treatments such as opioids and muscle relaxants. NSAIDs can also cause GI irritation, increase bleeding and platelet inhibition, worsen renal failure, and decrease the fusion rate of spinal instrumentation.

Subfascial pain catheters are FDA-approved or FDA-cleared devices which can address post-operative pain through the introduction of subfascial catheters that deliver a constant infusion of an anesthetic medication into the surrounding post operative paravertebral musculature. Such catheters may be silver impregnated, decreasing infection rates.

There are several types of neurotoxin proteins described in the literature which could be a potential substitute for Botox. Our description will mention Botox but is not limited to Botox. The Botulinum toxin is a neurotoxin protein produced by the bacterium Clostridium botulinum. In December 1989, BTX-A (Botox®) was approved by the U.S. Food and Drug Administration (FDA) for the treatment of strabismus, blepharospasm, and hemifacial spasm. In April 2002, the FDA announced the approval of botulinum toxin type A (Botox Cosmetic®) to temporarily improve the appearance of moderate-to-severe frown lines between the eyebrows (glabellar lines). BTX-A has also been approved for the treatment of excessive underarm sweating. There has been some more recent use of BTX-A for the treatment of spasticity and muscle pain disorders, with approvals pending in many European countries and studies on headaches (including migraine), prostatic symptoms, asthma, obesity and other indications ongoing. Botox® is manufactured in the U.S. by Allergan, Inc. for both therapeutic as well as cosmetic use. Botulinum Toxin Type B (BTX-B) received FDA approval for treatment of cervical dystonia in December 2000. Trade names for BTX-B are Myobloc® in the United States, and Neurobloc® in the European Union.

There are seven serologically distinct toxin types, designated A through G; 3 subtypes of A have been described. The toxin is a two-chain polypeptide with a 100-kDa heavy chain joined by a disulfide bond to a 50-kDa light chain. This light chain is an enzyme (a protease) that attacks one of the fusion proteins (SNAP-25, syntaxin or synaptobrevin) at a neuromuscular junction, preventing vesicles from anchoring to the membrane to release acetylcholine. By inhibiting acetylcholine release, the toxin interferes with nerve impulses and causes flaccid (sagging) paralysis of muscles in botulism as opposite to the spastic paralysis seen in tetanus. It was discovered in the 1950s that injecting overactive muscles with minute quantities of botulinum toxin type-A decreased muscle activity by blocking the release of acetylcholine at the neuromuscular junction, thereby rendering the muscle unable to contract for a period of time.

BOTOX® (Botulinum Toxin) is indicated for the treatment of cervical dystonia in adults to decrease the severity of abnormal head position and neck pain associated with cervical muscular spasms. BOTOX® is also indicated for the treatment of severe primary axillary hyperhidrosis that is inadequately managed with topical agents. Finally, BOTOX® is indicated for the treatment of strabismus and blepharospasm associated with dystonia, including benign essential blepharospasm or VII nerve disorders in patients 12 years of age and above.

BOTOX® temporarily stops muscle spasms in a non sedating manner. It also has been reported to have secondary effects of lessening the sensation of pain.

U.S. Pat. No. 6,447,787 teaches methods for enhancing would healing.

U.S. Pat. No. 3,966,934 teaches synergistic local anesthetic compositions.

U.S. Pat. No. 4,029,793 teaches synergistic local anesthetic compositions.

United States Patent Publication No. 2008/0292612 teaches a composition containing several botulic toxins.

United States Patent Publication No. 2007/0264373 teaches toxin induced sympathectomy.

United States Patent Publication No. 2009/002890 teaches a process for providing a temperature-stable muscle relaxant on the basis of the neurotoxic component of botulinum toxin in a solid form.

United States Patent Publication No. 2008/0213315 teaches clostridial toxin pharmaceutical compositions.

United States Patent Publication No. 2008/0021051 teaches phycotoxins and uses thereof.

United States Patent Publication No. 2007/0026019 teaches Botulinum toxin compositions.

The present invention seeks to combine the beneficial effects of a local anesthetic compounds (such as lidocaine) administered through a catheter-based device and injection of a neurotoxic protein compound (such as Botulinum toxin) for the treatment of post-operative and chronic pain in patients. The invention provides an improved post-operative and chronic pain management strategy, which decreases post-operative and chronic pain, increases patient mobility, decreases the duration of hospital stays, and decreases the overall medical costs incurred, improving economic recuperation and improving overall patient satisfaction.

### SUMMARY

The present invention provides for systems, and methods for the use of such systems, that allow for the administration of a sustained release of a local anesthetic compound (such as, but not limited to, lidocaine or bupivicaine) through a catheter-based administration system in combination with direct injection of a neurotoxic protein compound (such as Botulinum toxin) for post-operative and refractory muscular pain. The systems utilize specific catheter-based administration protocols and methods for placement of the catheter in association with muscles surrounding the spine. The utilization of an initial bolus of a specific combination of medications (local anesthetic compound and/or neurotoxic protein compound) followed by a dosage pump administration through the catheter is anticipated. A variety of local anesthetics (in addition to lidocaine or bupivicaine) and a number of different neurotoxin proteins (in addition to the Botulinum toxin mentioned above) may be utilized in the medicament administration protocols described. It is the combination of these two interventions that is a novel idea in providing improved pain relief in a non-sedating manner as an alternative to current treatments to chronic and post operative pain treatments.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a partially schematic diagram showing a system for administering the medicament combination associated with the methods in an embodiment of the present invention; and

FIG. 2 is a flow chart diagram describing the basic steps in the method of administration of the compound of the system in an embodiment of the present invention.

### DETAILED DESCRIPTION OF PREFERRED EMBODIMENTS

The present invention relates to systems and methods for treating pain by delivering a local anesthetic concurrently with a neurotoxin protein. Pain may result from a surgical or muscle splitting procedure, or may be refractory to conventional treatment as in chronic pain symptoms. In a preferred embodiment, the local anesthetic may be lidocaine or bupivicaine, and the neurotoxin protein may be Botulinum toxin. In a preferred embodiment of the invention, the local anesthetic may be delivered via a catheter system, and the neurotoxin protein may be delivered via direct or percutaneous injection.

In a preferred embodiment, the present invention may comprise a molecule which inhibits the release of acetylcholine at a neuromuscular junction combined with a local anesthetic. The molecule which inhibits the release of acetylcholine at a neuromuscular junction may be a neurotoxin protein, preferably a Botulinum toxin, preferably BTX-A, BTX-B, BTX-C, BTX-D, BTX-E, BTX-F, BTX-G, most preferably BTX-A or B. In a preferred embodiment, the molecule which inhibits release of acetylcholine at a neuromuscular junction may be delivered concurrently with a local anesthetic, preferably lidocaine or bupivicaine, and administered interior to the fascia of a subject. Neurotoxin proteins appropriate for use in the present invention include any toxin that acts specifically on nerve cells, especially by interacting with membrane proteins such as ion channels, including: Agitoxin, Batrachotoxin, Botulinum toxin, Calcicludine, Calciseptine, Charybdotoxin, Domoic acid, Hefutoxin, Kokoi venom, Margatoxin, Maurotoxin, PhTx3, Saxitoxin, Scyllatoxin, Slotoxin, Taicatoxin, and Tetrodotoxin.

In a preferred embodiment, the invention may comprise a molecule which inhibits the release of acetylcholine at a neuromuscular junction concurrently with a local anesthetic, preferably an ester or amide based local anesthetic. In a preferred embodiment, the local anesthetic may be lidocaine, prilocaine, bupivicaine, levobupivacaine, ropivacaine, or dibucaine, most preferably lidocaine or bupivicaine. Local anesthetics appropriate for use in the present invention include esters, for example Benzocaine, Chloroprocaine, Cocaine, Cyclomethycaine, Dimethocaine, Larocaine, Propoxycaine, Procaine, Novocaine, Proparacaine, Tetracaine, and Amethocaine; amides, for example Articaine, Bupivacaine, Carticaine, Cinchocaine, Dibucaine, Etidocaine, Levobupivacaine, Lidocaine, Lignocaine, Mepivacaine, Piperocaine, Prilocaine, Ropivacaine, Trimecaine; and naturally occurring anesthetics including Saxitoxin, and Tetrodotoxin.

The present invention contemplates the use of a Botulinum toxin at a dose of between 1 unit and 900 units, administered in a concentration of 0.001 units/kg-30 units/kg. Preferably the Botulinum toxin will be administered at a dose of between 10 units and 350 units, administered in a concentration of 0.01 units/kg-20 units/kg, most preferably at a dose of 20-200 units, and in a concentration of 1 units/kg-10 units/kg.

The present invention contemplates the use of an amide based local anesthetic at a therapeutically effective dose, preferably between 0.01% and 8.

In a preferred embodiment, the invention may comprise a method for treating chronic pain. The pain maybe in a subject that has undergone a surgical or muscle splitting procedure, or the pain may be refractory or chronic pain in a subject that has undergone one or more treatments for pain, such as massage, physical therapy, occupational therapy, or pain management treatments including epidural facet, or intramuscular injections. In a preferred embodiment, the invention may be used to treat chronic pain or use to treat pain in a subject that has undergone surgery, including neurosurgery, spinal surgery, a caesarian section, joint surgery, abdominal surgery, orthopedic surgery, general surgery, cardiothoracic surgery, obstetric or gynecologic surgery, plastic surgery, podiatric surgery or any type of muscle splitting surgery. It is contemplated that the present invention may provide treatment for pain derived from incisions, bruises, inflammatory responses, general tenderness, and muscle overuse, as well as pain derived from muscle spasm or those from chronic or refractory pain.

In a further preferred embodiment, the present invention may comprise a method for treating: acute and chronic pain syndromes, muscle strains and sprains, tendon and ligament injury, fracture related pain, post operative pain, causalgia/reflex sympathetic dystrophy, radiculopathic pain, muscle spasms, myofascial pain syndromes, plexopathy pain syndromes (i.e. brachial, sacral), rheumatologic or immunologic diseases, spinal stenosis, spondylosis, spondylolesthesis, rotator cuff pathology, muscle dystonia, fibromyalgia, cerebral palsy, hemifacial spasm, rheumatoid pathology, osteoarthritis, neuropathic pain syndromes, musculoskeletal pain disorders and syndromes, back and neck pain, cauda equina syndrome, crush injuries, and other related disorders.

The present invention may represent: an alternative to conventional pain treatment regiments; a pain control mechanism for post surgical use including pain secondary to muscle splitting procedures; a pain management strategy for pain which is refractory to minimally invasive pain management regiments including percutaneous epidural steroid injections, percutaneous facet injections, percutaneous muscle injections with lidocaine or other general anesthetic; and a treatment option for pain which is refractory to conventional pain management strategies.

In a preferred embodiment, the neurotoxin protein may be administered directly into the muscle, preferably via injection. The local anesthetic may be administered continuously, preferably via a catheter which is connected outside the skin to a reservoir of the local anesthetic.

In a preferred embodiment, the invention utilizes an administration system that includes a catheter that is placed within the patient and is supplied with a local anesthetic from an infusion pump or a drip administration system at a regulatable dosage. A neurotoxin protein is concurrently administered by subfascial direct injection. In a preferred embodiment, the catheter is placed subfascially, or intramuscularly. The catheter preferably delivers medication below the fascia.

The present invention further contemplates both subfascial administration of a neurotoxin protein, and suprafascial administration such that the neurotoxin can be indirectly delivered to the subfascial space. The invention includes any mechanism for delivering a neurotoxic protein to the subfasical space, including subfasical injection, or other forms of subfasical administration, and also suprafascial injection, or other forms of suprafascial administration which may result in delivery of neurotoxin protein to the subfasical space.

In another preferred embodiment, the invention utilizes an administration system which includes a percutaneous injection of neuroprotein to the subject, preferably below the fascia, preferably by a pain management physician, chiropractor, anesthesiologist, neurosurgeon, general surgeon, interventional radiologist, orthopedic surgeon, or a family practitioner. A subfacial intramuscular catheter which is connected to a reservoir of local anesthetic may then be placed in the subject.

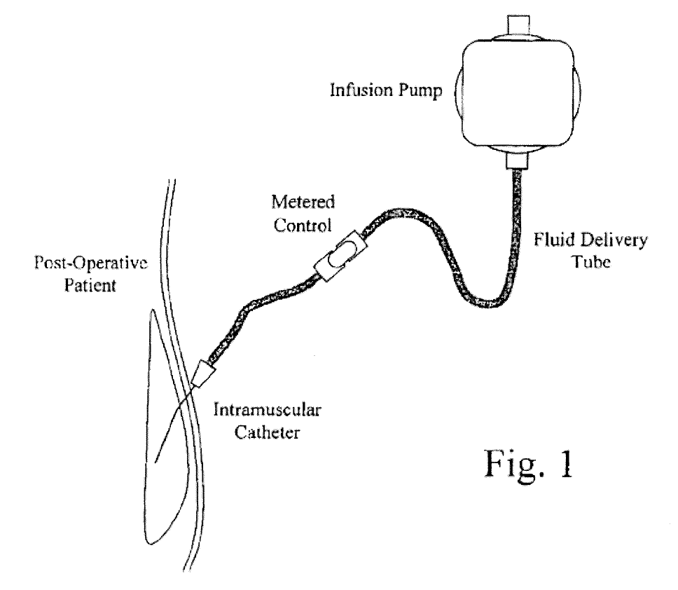

FIG. 1 shows the intramuscular placement of the catheter in an embodiment of the invention, although the invention contemplates alternate placements of similar catheters depending upon the specific surgical procedure that was carried out. In general, the system of the present invention finds optimal utilization in conjunction with post-operative patients recovering from surgical procedures, most typically those associated with the spine, abdominal, joint, obstetric/gynecologic or other muscle splitting operations.

In a preferred embodiment, the invention comprises administration of a local anesthetic through an infusion catheter, preferably placed such that the anesthetic is delivered subfascially. The anesthetic is delivered concurrently with a neurotoxin protein, which is administered by direct injection. An exemplary system that may be modified for use in conjunction with the methods of the present invention is available through I-Flow Corporation under the ON-Q® Painbuster ball® infusion catheter. Although the referenced system is directed to epidural placement of the catheter, the basic components of the system are operable in conjunction with the methods of the present invention. This system can be modified to provide subfascial or intramuscular delivery.

An exemplary protocol associated with the described system and which is appropriate for the administration of compounds in the present invention may be described as follows. An initial bolus of medications as described below is administered via an intramuscular catheter to the surgical region of the patient. In a preferred embodiment, the bolus of medications may be delivered subfascially. For example, an effective amount of local anesthetic, preferably lidocaine or bupivicaine, may be delivered subfascially, and a neurotoxin, preferably Botulinum toxin, may be combined in a normal saline solution and introduced according to the method of the present invention.

A further exemplary protocol associated with the described system and which is appropriate for the administration of compounds in the present invention may be described as follows. An initial bolus of neurotoxin protein is injected subfascially into the subject. The subject is then fitted with a catheter that is placed within the subject and is supplied with a local anesthetic, preferably from an infusion pump or a drip administration system at a regulatable dosage.

The present invention further contemplates a diagnostic tool for differentiating between pain resulting from muscle abnormalities and pain resulting from skeletal abnormalities, such as osteoarthritis, facet degeneration or other bon) abnormalities. A local anesthetic and a neurotoxin protein may be administered to a patient experiencing pain. If the pain improves, the pain is suspected to originate in the muscle, and the patient may continue treatment for an appropriate period of time, preferably approximately 6 months. In a preferred embodiment, the patient may receive the local anesthetic for a period of approximately 5-7 days after beginning treatment, and may receive the neurotoxin protein periodically for approximately 6 months, preferably every 4 to 6 weeks. If the patient does not improve using the treatment of the present invention, the pain is suspected to be related to joint pain, and other appropriate action can be taken more quickly as a result of this diagnosis. Therefore, the present invention may serve as a diagnostic tool for differentiating patients with a bone abnormality, such as might benefit from epidural or facet joint injections, or muscular pain, which would benefit from treatment according to the present invention as described by the mechanism of action of Botox.

Without wishing to be bound by theory, it is contemplated that the local anesthetic numbs the muscular pain after subfascial injection or delivery by a catheter. The molecule which inhibits the release of acetylcholine at a neuromuscular junction may then paralyze the muscle after percutaneous administration or direct intra muscular injection by direct visualization. Both medications may be administered in low doses intramuscularly such that they do not affect the entire muscle. It is contemplated that the paralytic effect causes less spasm of the muscle and therefore lessens the pain a patient or subject feels related to muscle spasm. Moreover, it is contemplated that the sensation of pain may be lessened by administration of the molecule which inhibits the release of acetylcholine at a neuromuscular junction, because inhibition of the release of acetylcholine from the nerve endings may decrease nerve impulses or transmission.

The present invention further contemplates that pain in a subject may be managed in distinct stages. Within 3 to 5 days of beginning treatment, a subject may receive local anesthetic via catheter which is fixed within the subject. This causes numbness of the muscle during this time. A neurotoxin protein, preferably Botulinum toxin, may be administered immediately or within the first 5 days of beginning treatment by direct injection. This functions to decrease muscle spasm and the sensation of pain. Second, within 5 days to 6 weeks of beginning treatment, administration of anesthetic may be ceased while the neurotoxin protein continues to exert effects of decreasing muscle spasm and sensation of pain. It is contemplated that decreasing muscle spasm during recovery may speed recovery by aiding in healing, and caused decreased scar formation. In a preferred embodiment, a subject may recover post-operatively without having muscle spasm for approximately 4-6 weeks.

The catheter in the system for use with the present invention is first secured at the insertion site with an appropriate dressing to prevent catching or pulling secure tubing from a lateral position on the back with tape or steri strips. The pump may be externally supported (as with a convalescing patient) or may be secured to the patient on a carrying clip. Such systems allow the patients to sponge bathe while the catheter is in place, although the wound site should not be subjected to bathing, showering, or swimming. Light activity is recommended for the patient until the catheter is removed.

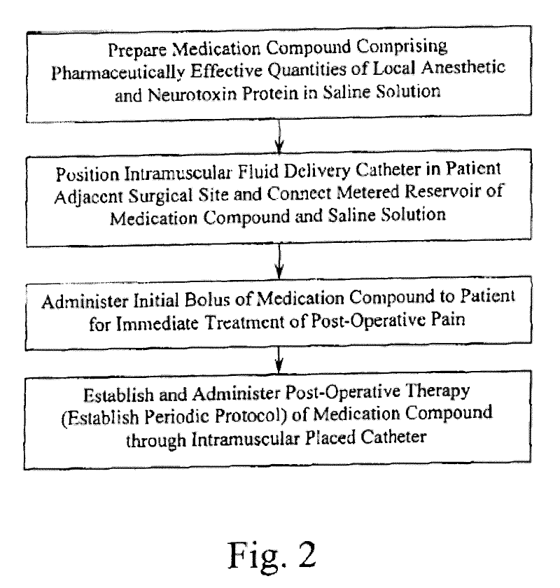

FIG. 2 provides the basic method steps associated with the use and administration of the medication compound of the present invention. In the initial step, the medication compound comprising pharmaceutically effective quantities of the local anesthetic, such as lidocaine or bupivicaine, in saline solution is prepared. The second basic step in the process comprises positioning an intramuscular fluid delivery catheter in the post-operative patient adjacent to the surgical site and connecting a metered reservoir of the prepared medication compound. The third basic step in the established therapy is the administration of an initial bolus of the local anesthetic to the patient for the immediate treatment of post-operative pain. The initial composition and/or rate of administration may preferably be different for the initial bolus and the subsequent metered dosage. In a fourth basic step, a neurotoxin protein may be delivered, preferably by direct injection, either before, after, or during administration of the local anesthetic. Finally, the method of the present invention comprises the establishment and administration of an ongoing post-operative therapy which would, in the preferred embodiment, include a progressive reduction in the dosages of both the local anesthetic and/or the neurotoxin protein.

The catheter system and combination of a neurotoxin protein administered concurrently with a local anesthetic of the present invention may be used in any area throughout the body where subfascial or intramuscular administration can be accomplished. In a preferred embodiment, the invention is used in conjunction with a spinal-surgical procedure. In general, the system of the present invention finds optimal utilization in conjunction with post-operative patients recovering from muscle splitting surgical procedures, most typically those associated with the spine, abdomen, joint, and ob/gyn and other muscle splitting procedures.

The present invention further contemplates administration of a molecule which inhibits the release of acetylcholine at a neuromuscular junction combined with a local anesthetic via any method which can deliver the combination to the interior of a fascia of a subject. This may include topical, enteral, or parenteral, delivery, for example epicutaneous, intranasal, oral, intravenous, intraarterial, intramuscular, vaginal, intracerebral, intracerebroventricular, intracardiac, rectal, subcutaneous, intraosseous infusion, intradermal, intrathecal, intraperitoneal, intravesical, intracavernosal, transdermal, transmucosal, insufflational, sublingual, buccal, inhalational, intracisternal, epidural, or intravitreal administration. In a preferred embodiment, delivery may occur via a catheter system placed subfascially or intramuscularly within a subject.

### EXAMPLE 1

Botulinum toxin in a dose of 1-2 units/kg is administered subfascially at the site of pain via direct injection to a subject experiencing pain. A catheter is placed subfascially at the site of pain within the subject and is supplied with bupivicaine at a concentration of 0.5%, from an infusion pump at a dosage of 2 ml/hr.

With wound open, the fascia and paraspinal muscles are identified. The length of catheter needed is estimated. A trocar is placed 2 cm lateral to the midline incision. The trocar is bent to allow placement of catheters past fascia and into the paravertebral muscles. Catheters are placed in the thoracic and lumbar spine (rostral to caudal insertion) or cervical spine (caudal to rostral insertion). Catheters are secured with steristreps and dermabond or tegaderm. The procedure is repeated on the opposite side.

### EXAMPLE 2

A subject undergoes spinal surgery. After the procedure is complete, the surgeon locates the fascia. The surgeon bends the trocar, and inserts the trocar into paraspinal muscles. The surgeon threads the catheter, and holds the catheter with non-tooth forceps against the skin. The surgeon pulls and peals tips of plastic equally in opposite directions.

Botulinum toxin in a dose of 1-2 units/kg is administered subfascially at the site of pain via direct injection to the subject experiencing pain. A catheter is placed subfascially at the site of pain within the subject and is supplied with bupivicaine at a concentration of 0.5%, from an infusion pump at a dosage of 2 ml/hr.

### EXAMPLE 3

A patient with muscle or back pain which does not respond to conventional treatment is fitted with a catheter which administers bupivicaine subfascially. The patient also receives an injection of Botulinum toxin subfascially. Bupivicaine is administered via the catheter at a concentration of 0.5% for a period of 5-7 days. Botulinum toxin is administered via injection at a concentration of 1-2 units/kg at the beginning of treatment, and periodically at intervals of approximately 4-6 weeks for a total treatment time of approximately 6 months.

### EXAMPLE 4

A subject experiencing pain is treated with bupivicaine at a concentration of 0.5% administered through the catheter at a rate of 2 ml/hr for a time period of 5 days. The subject also receives an injection of botulinum toxin at a concentration of 1-2 units/kg. The pain improves using this treatment, and bupivicaine treatment is discontinued after 7 days, and the subject receives further injections of botulinum toxin every 6 weeks for a total period of 6 months.

### EXAMPLE 5

A subject experiencing pain is treated with bupivicaine at a concentration of 0.5% administered through the catheter at a rate of 2 ml/hr for a time period of 5 days. The subject also receives an injection of botulinum toxin at a concentration of 1-2 units/kg. The pain does not improve, and the subject begins treatment for joint pain.

Although the present invention has been described in terms of the foregoing preferred embodiments, this description has been provided by way of explanation only and is not intended to be construed as a limitation of the invention. Those skilled in the art will recognize modifications of the compounds, systems, and methods of the present invention that might accommodate specific surgical and post-operative requirements with the patient. As indicated above, the specific local anesthetic and the specific neurotoxin protein may be varied in their composition ratios and dosages. In addition, the specific manner of administration, including the initial administration and the metered administration, may be varied according to the mobility of the patient and the quantities of medication required. These modifications do not necessarily depart from the spirit and scope of the methodology of the present invention.

### EXAMPLE 6

### Intraoperative Placement of Infusion Pain Catheters into the Paraspinal Muscles and not into the Lumbar Fascia

A 57 year old presented with progressive numbness, tingling and weakness of bilateral arms and progressive gait difficulties. After further investigative studies he was diagnosed with cervical myelopathy secondary to cervical stenosis, cervical spinal cord contusion with radiographic cord signal abnormalities. Because most of his compression was posterior, a C3-7 posterior cervical laminectomy and fusion was done to decompress the spinal cord.

### Intraoperative Description

After decompression of the spinal cord, placement of both lateral mass screw instrumentation, and the posterior lateral arthothesis, the pain catheters were placed. Placement of the catheters was done in the following way: From the midline incision, two five inch silver impregnated catheters, which deliver 2 cc/hr of Marcaine 2% without epinephrine was introduce bilaterally 2 cm lateral and inferior of the inferior end of the wound. Trocar needle which are covered with a disposable plastic sheath were fashioned to be introduced into the paraspinal cervical muscles. Direction of the trocar needles were aided by shaping of the catheters and by feeling the catheters midline with my thumb as it progressed thru the paraspinal muscles. Visual inspection of each side of the midline incision was done to verify that no part of the plastic sheath covering the trocar needles could be seen thru the paraspinal muscle.

Once the trocar needles were positioned into the paraspinal muscles, the trocar needles were removed and the plastic sheath was left in place. Then silver impregnated infusion catheters were introduced thru the plastic sheath where the trocar needles had been. As the infusion catheters reached the end of the plastic sheath, resistance from the paraspinal muscles prevented the infusion catheter from progressing any further.

Holding the infusion catheters against the skin, the plastic sheaths were peeled off the infusion catheters bilaterally. This was accomplished because the plastic sheath has two tabs at the portion exposed at the skin which split in two as the plastic sleeve was elevated out, leaving behind only the infusion catheters. The infusion catheters were then attached to a 400 cc reservoir containing Marcaine 0.5% without epinephrine.

### Postoperative Course

On post operative day 0, the patient was ambulating the halls of the hospital and participating in occupational and physical therapy evaluations. On postoperative day 3, the patient's pain was sufficiently controlled to be discharged home. He rated his pain 3-4/10 on the visual analog pain scale and did not require any patient controlled analgesic (PCA) administration during his hospitalization.

On post op day #15 the patient was seen for suture removal without signs of infection of the previous infusion catheter sites. There were no markings noted were the infusion catheters had been.

### EXAMPLE 7

A 66 year old male was suffering from significant progressive low back and radiating leg pain. On further diagnostic work up he was noted to have lumbar levoscoliosis with coronal imbalance and the apex of the imbalance at L4-5. The MRI of the lumbar spine demonstrated significant bilateral foramenal compression of the L4-S1, loss of disk height and significant disk degeneration of L5-S1. After discussion with the patient, a L4-S1 decompressive laminectomies with bilateral foraminotomies, L5-S1 transforaminal interbody fusion and coronal correction of his anatomical imbalance with pedicle instrumentation from L3-S1 with posterior lateral arthodesis from L3-S1 was proposed.

### Intraoperative Description

After decompression of the central canal of L4-S1 with bilateral foramenotomies, L5-S1 transforaminal interbody fusion and correction of the coronal imbalance was accomplished, the pain catheters were then placed. Placement of the catheters was done in the following way: From the midline incision, two five inch silver impregnated catheters, which deliver 2 cc/hr of Marcaine 2% without epinephrine were introduce bilaterally 2 cm lateral and inferior of the superior end of the wound. Trocar needles covered with a disposable plastic sheath were fashioned to be introduced into the paraspinal lumbar muscles. Direction of the trocar needles were aided by shaping of the catheters and by feeling the catheters midline with the surgeon's thumb as it progressed thru the paraspinal muscles. Visual inspection of each side of the midline incision was done to verify that no part of the plastic sheath covering the trocar needles could be seen thru the paraspinal muscle.

Once the trocar needles were positioned into the paraspinal muscles, the trocar needles were removed and the plastic sheath was left in place. Then silver impregnated infusion catheters were introduced through the plastic sheath where the trocar needles had been. As the infusion catheters reached the end of the plastic sheath, resistance from the paraspinal muscles prevented the infusion catheter from progressing any further.

Holding the infusion catheters against the skin, the plastic sheaths were peeled off the infusion catheters bilaterally. This was accomplished because the plastic sheath has two tabs at the portion exposed at the skin which split in two as the plastic sleeve was elevated out, leaving behind only the infusion catheters. The infusion catheters were then attached to a 400 cc reservoir containing Marcaine 0.5% without epinephrine.

### Post Operative Course

On post operative day 1 the patient was ambulating with physical therapy and the PCA was discontinued. On post operative day 3 the patient was accepted to rehab and transferred to rehab on post operative day 4. The patient had rated his pain level at a 5-6/10 on the visual analog pain scale on POD#1 and on the day of discharge rated his pain as 4/10 and well controlled with pain medications.

### REFERENCES CITED

The following references, to the extent that they provide exemplary procedural or other details supplementary to those set forth herein, are specifically incorporated herein by reference.

### U.S. PATENT DOCUMENTS

U.S. Pat. No. 6,447,787 to Gassner et al., issued Sep. 10, 2002;

U.S. Pat. No. 3,966,934 to Adams, et al., issued Jun. 29, 1976;

U.S. Pat. No. 4,029,793 to Adams, et al., issued Jun. 14, 1977;

United States Patent Publication No. 2008/0292612 with Berrutet listed as the inventor, published Nov. 27, 2008;

United States Patent Publication No. 2007/0264373 with Carroll et al. listed as the inventors, published Nov. 15, 2007

United States Patent Publication No. 2009/0028906 with Grein et al. listed as the inventors, published Jan. 29, 2009

United States Patent Publication No. 2008/0213315 with Hunt listed as the inventor, published Sep. 4, 2008

United States Patent Publication No. 2008/0021051 with Wilson listed as the inventor, published Jan. 24, 2008

United States Patent Publication No. 2007/0026019 with Hunt listed as the inventor, published Feb. 1, 2007

1-5. (canceled)

6. A method for treating chronic and post-operative pain, comprising the steps of:
a) administering a neurotoxin protein subfascially to a subject; and
b) administering a local anesthetic subfascially to a subject.

7. The method of claim 6, wherein administration of the neurotoxin protein is via injection.

8. The method of claim 6, wherein administration of the anesthetic is via a catheter.

9. The method of claim 6, wherein either the neurotoxin protein or the local anesthetic are administered indirectly to the subfascial space and allowed to diffuse into the subfascial space.

10. The method of claim 6, wherein administration of the neurotoxin protein is directly or percutaneously into a muscle of the subject.

11. The method of claim 6, wherein the local anesthetic is lidocaine or bupivicaine.

12. The method of claim 11, wherein the lidocaine is at a concentration of between 0.01% and 8%.

13. The method of claim 11, wherein the bupivicaine is at a concentration of between 0.01% and 8%.

14. The method of claim 6, wherein the neurotoxin is Botulinum toxin.

15-20. (canceled)
